(19) United States
(12) Patent Application Publication
(10) Pub. No.: US 2016/0045473 A1
(43) Pub. Date: Feb. 18, 2016

(54) Biological Basis Of Adult Wellness

(71) Applicant: Rory Powell Blake, Raleigh, NC (US)

(72) Inventor: Rory Powell Blake, Raleigh, NC (US)

(21) Appl. No.: 14/850,417

(22) Filed: Sep. 10, 2015

Related U.S. Application Data
(63) Continuation-in-part of application No. 13/956,327, filed on Jul. 31, 2013, now abandoned.
(60) Provisional application No. 61/741,990, filed on Aug. 1, 2012.

(51) Int. Cl. A61K 31/401; A23L 1/305

(52) U.S. Cl. CPC A61K 31/401; A23L 1/3051; A23V 2002/00

(57) ABSTRACT

A composition for a dietary supplement. The supplement may include: (a) an effective amount of amino acid and a biocompatible solvent, wherein the effective amounts of amino acid is between about 1 gram per day and about 20 grams per day; and (b) a food additive to aid in administration.

### CROSS-REFERENCE TO RELATED APPLICATIONS

This application is a continuation-in-part of U.S. Ser. No. 13/956,327, filed Jul. 31, 2013, which claims priority of Provisional Application Ser. No. 61/741,990 filed Aug. 1, 2012, the contents of which are hereby incorporated by reference in its entirety.

### FIELD

The present inventions relate generally to nutrition and, more particularly, to improvement in the health of individuals by correction of nutritional uptake imbalance.

### BACKGROUND

In nature, the longest-lived mammals are of the larger species. Genetically, they make a larger investment in gestation. The genetic payback is a larger and more sophisticated animal. With an increase in size comes a need for an increased capacity to absorb nutrients. Larger animals intrinsically have a much greater need for nutrients, both to supply energy—and for the maintenance of their much greater bulk. Without both the availability of additional nutrition and an additional capacity to absorb this additional nutrition, the animal would go into a steady decline.

As it is programmed into their genes, larger animals maintain an intrinsic capacity to uptake more nutrients than do smaller species. They can also retain larger reserves of critical nutrients. Just as printer cartridges may be quickly emptied with heavy use or more slowly depleted with lighter use, this genetic capacity for greater intake can be reconfigured. It may proceed normally, or if need be, configured into a lower daily intake over a longer period of time.

In addition to size, the longest living mammals in nature have in common being at the top of the food chain. The longest living animals share a characteristic of not being constantly under physically and emotionally stress. Synthesis of signaling peptides, as is required to produce neural hormones, takes away from critical amino acid reserves. All animal groups exhibit individuals within their species attaining a great age. Within any species, environmental modifications to ease the individual's circumstances can extend that individual's lifespan due to prolongation of critical amino acid reserves.

The success of this strategy is correlated with the species genetic potential for physical size. The potential for larger size carries with it the potential to ingest and to absorb more nutrients. This becomes an increased potential to be able to continue forward. The animal who can continue forward with somewhat lowered quantities of nutrients will then continue forward over a much longer period of time.

In England, a goldfish, confined to a small bowl was reported to have lived for over 40 years. A goldfish characteristic is that they do not grow to their maximum size when confined to a small environment. Individuals, such as this goldfish, however, always retain some amount of this unused genetic capacity. This unused nutritional uptake capacity is what then becomes the extra capacity to continue forward.

In humans, the smaller female tends to live a few years longer than the typically much larger or much more physical male of the species. In general, a female eats somewhat less than a male. This is especially true if a male is employed in a highly physical occupation, such as that of manual labor. Heavy, manual labor requires the uptake and expenditure of more energy. Given the same generic capacity for the intake and absorption of nutrients, the female would be able to intake smaller meals over a much longer period of time before reaching the species-specific intake limit. Sharing a smaller size within a larger species would seem to aid in both health and wellness. A smaller body mass with the retention of the larger species-specific capacity for absorbing nutrition is responsible for the above life-extending phenomenon.

In France, long leisurely meals and the occasional bon-bon for a snack are highly prized traditions. Spreading nutritional intake equally over the day has long been thought to be quite beneficial to your metabolism and health. Presenting smaller amounts of nutrition continually, rather than massive quantities (and those all at once) appears to aid retention of nutrient uptake capacity. Drinking wine with meals is another French custom. The tradition is thought to aid in digestion. This may be important to nutrient uptake and longevity, as well. Living to the age of one hundred and twenty-two years, a French female holds the record for the longest life attained by a human.

Metabolism has long been known to be a factor in health and wellness. Within a species, a more efficient metabolism can lead to a much longer lifespan. Disorders of metabolism such as metabolic syndrome and diabetes are clearly shown to damage health and to shorten lifespan. Maintaining a healthy metabolism is key to health. Many and various studies affirm this. Trials with the fruit fly have illustrated that improving various aspects of intestine function can increase lifespan by as much as forty percent. Caloric restriction has been shown as being effective in prolonging lifespan within all animal species.

Caloric restriction was before thought to be effective in prolonging an individual's lifespan because it limits expenditures of energy and the resulting “wear and tear” on the body. The more important principle of this disclosure also applies. Caloric restriction works primarily by maximizing a species genetically predetermined capacity for nutrient uptake. Not overwhelming these uptake receptors and, in fact, not overwhelming the digestive system with excess food will improve health and increase survival.

The limiting aspect of lifespan expansion is the ability to uptake and to utilize nutrients. Elephants live a long time. Most could live even longer. However, many elephants ultimately succumb to starvation—simply due to their teeth wearing down.

In biological systems, prevention of wear preserves bodily resources. Avoiding wear conserves the resources and the nutritional reserve that would otherwise be needed to accomplish repairs. Conservation of resources, such as the bodily raw materials required for ordinary repair, is especially beneficial in older individuals. Recovery time after exercise, injury, or surgical procedures becomes much longer as individuals age. Simply put, older individuals who can stay healthy will live longer. Those who escape disease or serious injury will always outlive those survivors of major surgeries or those who have frequently become injured.

In the oldest of adults, extremely slow recovery from even the most minor bodily insult is common. The limiting factor in senior adult healing is not a simple slowdown in the mechanisms of cellular repair. The limiting factor is in the depletion of bodily reserves. The very old suffer from a generalized lower level of stored critical nutrients, the very nutrients essential for bodily repair. There are two equally important and closely related factors at work within this disclosure. Slower healing is due from both a diminished capacity to absorb critical nutrients from the gut and to the body's need to cannibalize existing structures to obtain the needed repair materials. The inability to intake nutrients ultimately leads to a generalized shortage of raw materials within the body and the lack of easily available materials that can be used for repair.

In extreme aging, the nutritional imbalance mimics the process of starvation. The aged body and a body dealing with starvation are alike in many important ways. In starvation, the body has an orderly process of shutting down non-vital functions to keep the individual alive until the current crisis is over. Neural hormones are the peptide structures that serve as messengers in the body. In the human body, nothing good will happen without something telling it to happen. The level of hormone decline is well documented in aging. The order of hormonal decline and loss of function is virtually indistinguishable in both systems, if not as well studied in starvation. Many processes of aging and of starvation are the same, if only slowed during the aging process.

Our bodies will not just “quit” when faced with diminished resources. A precise order of importance in resource utilization comes into play. In extreme aged individuals, the telomeres' of genes become shorter—not as a result of genetic damage. Telomeres' become foreshortened because in starvation mode the seemingly extra component is critically needed elsewhere. Changes in the genes are not a primary cause of aging. In this case, nutritional imbalance may be a cause of this change to the genes. In the interest of staying alive, the body will reabsorb all rarely used and less essential bodily components; including those immediately less important structures within a gene.

There are twenty amino acids comprising both bacterial and individual human cells composing the human body. The species-specific nutrient uptake limit is essentially a limit on the amount and kind of amino acids that may be absorbed by a multi-cellular organism during its lifetime. This lifetime limit will impact the single-cell bacteria that happen to be living in the gut of the larger organism, as well.

Serious scholarly work in basic science was halted by the many pressures on academia resulting from the outbreak of World War II. The field of amino acid science had been very fruitful prior to the war, with most of our knowledge, today, being based on work published during that period. The last truly novel work was published in 1942, but the included work was mostly done pre-war. After the war, new challenges emerged—serving to crystallize the prior work as orthodoxy. That earlier work, today, remains accepted and unchallenged.

According to this orthodoxy, the eight “essential” amino acids are: isoleucine, leucine, lysine, methionine, phenylalanine, threonine, tryptophan and valine. The “non-essential” amino acids are: proline, alanine, arginine, asparagine, aspartic acid, carnitine, citrulline, cysteine, cystine, gamma-amino butyric acid, glutamic acid, glutamine, glycine, ornithine, serine, taurine, and tyrosine.

Other schemes, found in patent applications, further divide amino acids into: essential amino acids, e.g. isoleucine, leucine, lysine, methionine, phenylalanine, threonine, tryptophan, valine, or ilistidine, conditionally essential amino acids, e.g. tyrosine, cysteine, arginine, or glutamine, or non-essential amino acids, e.g. proline, glycine, alanine, serine, glutamic acid, aspartic acid, asparagines, taurine or carnitine.

Other patent disclosures have amino acids broken down as small non-polar, hydrophobic amino acids that include proline, glycine, alanine, leucine, isoleucine, valine, and methionine. The large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine and the negatively charged (acidic) amino acids include aspartic acid and glutamic acid.

In spite of these noted differences, the general assumption in current research is that all amino acids are absorbed in the same way. In this disclosure, the term “amino acids” as used herein, unless otherwise stated, refers to amino acids in free form and/or in salt form chosen from one of the above classes. In this disclosure, amino acids are accorded differing absorption characteristics in relation to these chemical and physical differences. This disclosure is of tile differing absorption of amino acids over time.

It has elsewhere been shown that metabolism and repair becomes less exact as an organism become older. The decline is real, if not the relationship to a cause. The observed decline may be due to a genetic change, a decline in nutrition or may be due to any of the multiple vectors of infectious disease. The decline noted may further be due to a combination of these factors. A physical decline eventually becomes noticeable with simple observation, even to the untrained eye. We may notice that we, physically, do not look as good nor do we move as well as we once did. We may notice that our bulk is increasing in some areas, or feel our metabolism is slowing. The first thought in these cases may be to eat less or to increase the amount exercise we are getting, as is advised in the prior art. Long term, neither course of action is certain to be therapeutic and, often, extremes of either may prove harmful.

The other area of decline, the decline in metabolic function, can be predicted, measured, and observed clinically. In a clinical setting, patients typically present first with hypertension, then metabolic syndrome, then “other” in a wide range of endocrine disorders. Hypertension causes “stroke” and metabolic syndrome can lead to diabetes. These disorders, however, can be treated clinically with success. The underlying cause of each of these syndromes is some “unknown” issue in the timely production of signaling hormones. This issue is a lack of the raw materials critical for peptide production.

Genetic changes that may, or may not, occur and what is broadly termed as “aging” are addressed elsewhere. This disclosure is of the mechanisms involved in the uptake of specific critical nutrients. Signaling peptides that control the feedback loops within the metabolism, as well as, the structural protein of rebuilding and repair have an important thing in common. In each process, the manufacture of protein is accomplished using specific component amino acids; each amino acid added to the next in a precise order. In both systems, proteins and peptides are precisely assembled combinations of among the twenty amino acids found within the body. The genes tightly control the entire process. If the body is lacking a specific amino acid, the organism cannot accomplish a specific metabolic process in a timely manner. If specific amino acids are in short supply, the body cannot accomplish specific repair. Without the exact amino acid necessary for the next step, and without said amino acid being provided at the exact time it is needed, it can be said that “nothing good will happen in the body.”

The resultant change in bodily structure and function is not a symptom of “aging.” This is not Alzheimer's disease, nor is it arthritis. This is the effect of a lack of protein nutrition. Further, it begins much earlier than the development of the traditional “aging” symptoms, as described by others. Early on, the body is able to cover for a lack of absorption or for low absorption of certain amino acids by consuming plentiful reserves. The body may, at first, simply be obtaining needed amino acids from areas where their loss is less visible, such as from the outside covering of blood vessels. The consumption of critical reserves for standard bodily maintenance and the continuation of normal function are the twin forces that combine to compromise health and wellness and to foreshorten longevity.

The prior art descriptive of decline as we grow older universally revolves around genetic change. This is because our genes are expected to maintain a tight control over the manufacture of protein within the body. One current and respected theory involves “tired genes.” It notes that some genes just wear out. Another theory places a limit on the number of times that a gene may be successfully copied. Other theories cite a culmination of genetic errors as the causes of the multitude of symptoms associated with that which is termed to be “normal aging.” A multiplicity of theories claim to account for the various symptoms associated with aging but none, thus far, account for the entire phenomenon.

No prior art on “Aging” takes into account, or can explain all of the changes that seem to be occurring, as we grow older. Interestingly, the current most popular theory on the causes of the physical and functional decline associated with growing older involves changes to an area of the gene that does not encode anything. Clearly, the generic component of the process deserves to be revisited.

In one disclosure, many and diverse symptoms are lumped together under the term “Ageing.” The same experiments are repeated again and again and the decline of genes with every replication is calculated with precision. The design of these experiments do not account for the “real world” variability of aging symptoms. What has actually been observed is quite different from that which is claimed.

There is another historical perspective. The post-World War progress made in penicillin and other antibiotics limited our concerns about bacteria. Other than diarrhea, a common side effect of antibiotics on our own beneficial bacteria, bacteria have become not of concern to us. Depending on how it is counted, there are ten to one-hundred times the number of bacterial cells living in or on our bodies, as there are cells in our own body. Bacteria living in the gut of an animal will play an important part in the nutrition of that animal. Humans are no exception to this natural law.

Young animals with “knock-out” genes that cause them to bear a physical resemblance to aging adults are not actual aged individuals. Their intestines are young intestines and these intestines are filled with active, young bacteria. These young animals may very easily benefit from the addition of low levels of nutrients that are difficult for older intestines populated with old and damaged bacteria to absorb.

Genetic change, as it relates to the longevity claims in prior art, needs to be addressed. The basis of this disclosure is contemporaneous genetic change is largely not responsible for the disease and the physical decline associated with growing older. As we grow older, many changes to processes under genetic control do occur. This is, again, why the genes are so often faulted. Genes, by themselves, are unable to cause every change that may or can occur. Much of the decline observed, both physical and metabolic, is due to a lack of absorption of specific nutrition. This lack of uptake leads to a depletion of bodily reserves and to the likely repeat of the observed error. The observation of this phenomenon is further compounded by the individual bodily responses to the lack of utility from the unmade protein. This accounts for the high variability of the signs and symptoms of disease observed within individuals as they begin to grow older.

Many of the observable and reported genetic transcription errors and omissions are due to the shortage of a specific amino acid needed to fully complete that specific process. This is why the progression of symptoms accompanying the attainment of a great age is more akin to the progression of change seen during the process of starvation. A slow starvation by the mechanism of a lack of intake and the depletion of bodily reserves is described in this disclosure.

Deleterious changes occurring in the body over time may be genetically related, disease vector related, or nutritional. Faulty processes occurring in the body due to nutritional factors may be effected by nutrition shortage and yet remain under genetic control. This direct genetic control may remain as genetically programmed, or the genetic code may have become altered due to damage or developmental changes. When a supply of the missing nutrition in an absorbable form is provided, proper function can be restored to the body. This would indicate, that during the period of the prior faulty processes, the genes had remained as originally programmed.

Thus, there remains a need for a new and improved a composition for a dietary supplement which addresses a wide range of health issues associated with poor diet, disease and aging while, at the same time, has very few adverse affects for most individuals.

### SUMMARY

The present inventions are directed to a composition for a dietary supplement. The supplement may include: (a) an effective amount of amino acid and a biocompatible solvent, wherein the effective amounts of amino acid is between about 1 gram per day and about 20 grams per day; and (b) a food additive to aid in administration.

In one embodiment, effective amount of amino acid is between about 2 grams and about 15 grams per day. In another embodiment, the effective amount of amino acid is about 10 grams per day. The amino acid may be L-proline.

In one embodiment, the biocompatible solvent is a polar organic solvent. The polar organic solvent may be an alcohol. The alcohol may be ethyl alcohol.

The concentration of amino acid, in a biocompatible solvent, may be between about 0.1 and about 0.5 grams per milliliter. In one embodiment, the effective concentration of amino acid in a biocompatible solvent is between about 0.2 and about 0.4 grams per milliliter. The effective concentration of amino acid in a biocompatible solvent may be about 0.3 grams per milliliter.

The dietary supplement may further include the balance water to aid in administration. In one embodiment, the balance water is more than about 50 vol. %. The concentration of water may be about 60 vol. %.

In one embodiment, the food additive is a flavoring. The flavoring may be a sweetener. The sweetener may be an artificial sweetener.

In one embodiment, the food additive is a colorant. The colorant may be Allura Red AC.

In one embodiment, the food additive is a texture additive. The texture additive may be a solid. The solid may be another food. The another food may be pulp from fruit or vegetables. The texture additive may also be a liquid. The liquid may be carbonated water.

The dietary supplement may further include stabilizers. In one embodiment, the stabilizers are selected from the group consisting of sorbic acid, ascorbic acid, sulfur dioxide, sodium benzoate and mixtures thereof.

Accordingly, one aspect of the present inventions is to provide a composition for a dietary supplement comprising an effective amount of amino acid and a biocompatible solvent.

Another aspect of the present inventions is to provide a composition for a dietary supplement comprising an effective amount of amino acid and a biocompatible solvent, wherein the effective amounts of amino acid is between about 1 gram per day and about 20 grams per day.

Still another aspect of the present inventions is to provide a composition for a dietary supplement comprising an effective amount of amino acid and a biocompatible solvent, wherein the effective amounts of amino acid is between about 1 gram per day and about 20 grams per day; and a food additive.

These and other aspects of the present inventions will become apparent to those skilled in the art after a reading of the following description of the disclosure when considered with the drawings.

### BRIEF DESCRIPTION OF THE DRAWINGS

FIG. 1 is a graphical representation of a response surface illustrating the relationship of administered dosage (in milliliters per day) and concentration of the dietary supplements as constructed according to the present inventions; and

FIG. 2 is a graphical representation of a response surface illustrating the relationship of normalized dosage (in grams of amino acid per day) and concentration of the dietary supplements as constructed according to the present inventions.

### DETAILED DESCRIPTION OF THE EMBODIMENTS

In the following description, like reference characters designate like or corresponding parts throughout the several views. Also in the following description, it is to be understood that such terms as “forward,” “rearward,” “left,” “right,” “upwardly,” “downwardly,” and the like are words of convenience and are not to be construed as limiting terms.

The present inventions are directed to a composition for a dietary supplement comprising an effective amount of amino acid and a biocompatible solvent. The expressed interest of this disclosure is the development of successful mechanisms to accomplish increased uptake or to correct an uptake imbalance of specific essential nutrients absorbed into the adult body. This decline in nutrient uptake can begin at differing times, but will occur in every adult individual. There can be great individual variability in the onset of this nutrient uptake decline and in the number and kinds of nutrients involved. The disease and the course of the diseases, which may occur without these additionally supplied nutrients, is highly individual. Without nutritional supplementation as described in this disclosure, the final response to a lack of critical nutrition is unmistakable. Nutritional needs will become harder and harder to meet until there is a total depletion of bodily reserves.

In some embodiments, the composition for a dietary supplement comprising an effective amount of amino acid and a biocompatible solvent includes an effective amount of amino acid greater than about 1 gram per day. In other embodiments, the effective amount of amino acid is between about 2 grams and about 20 grams per day; for instance, 10 grams per day.

The dietary supplement preferably includes the amino acid L-proline. In the embodiments disclosed below, the amino acid is dissolved in ethyl alcohol. However, the amino acid may be dissolved in other biocompatible solvents, such as other polar organic solvents. Biocompatible as used herein refers to a solvent that is deemed safe for human consumption.

Changes that begin with the lack of uptake of specific amino acids often take quite a long time to be physically evident, clinically significant, or even noticed. The body will attempt to find a way to provide for any “immediate” needs for amino acids. If specific amino acids are not taken in by adsorption, a way will be found to provide them for use in the body's processes. The process of amino acid recycling usually begins with the taking of needed amino acids from the more easily accessible areas of collagen. Simply “doing without” needed amino acids by stopping bodily processes is not an option. The body is designed to find a way to continue during lean periods. Doing without, becomes the final option.

Upon prolonged amino acid shortage, the body becomes more aggressive in breaking down what are considered less immediately important cells for their protein content. The amino acid defect will be made up. This can be accomplished by collection of the needed amino acids from different areas of the body. The individual manifestations are based on bodily criteria determining in what order the collection of the needed materials will proceed. Without nutritional uptake of the required critical amino acids—this process will proceed with certainty. If the deficit of specific amino acids continues for a long period, something else is certain. Apoptosis or cell death will increase and the body's protein mass will decline.

The adult body, at a certain point, begins a lowered absorption of nutrients from the digestive tract. More specifically, there occurs an evolutional, generically based, mechanical decline in nutritional uptake within the adult digestive tract. This decline in amino acid absorption begins much earlier than has been previously expected. Further, this decline will begin at different times and progresses at differing rates in different individuals.

A decreased mechanical absorption of certain specific amino acids over time is increasingly problematic. The body must have all 20 letters in its amino acid alphabet available, and available at the appropriate time, to form signaling peptides. The decline in absorption of specific amino acids may be a possible cause of common disorders such as hypertension and metabolic syndrome. These disorders are triggered by a lack of production of the peptides essential for communication between the involved organs.

The process is not fair. People who work hard all their lives simply cannot live as long as those who are able to take life relatively easily. We each have our own finite lifespan. Without uptake of critical nutrients, individuals who work in highly physical jobs quickly deplete bodily reserves. As they get older, the targeting of protein containing areas to obtain needed amino acids becomes much more ruthless. This process always decreases potential lifespan in order to obtain the amino acids needed for today. The object of the invention is the disclosure is of a method of prevention of the body digesting itself through improved nutrition. Health and wellness will flow from this of its own course. This disclosure makes no claim of correction, improvement, or having any effect whatever on genetic change, which may occur, or may not occur. This disclosure makes no claim of slowing or reversing or of the prevention of any natural process. The object of this invention is through specific improvements in the uptake of critical nutrition the window of good health can be held open, just a little longer.

Much is known today about the storage of fat and glucose within the body. Fat is stored in adipose tissue and glucose is stored as glycogen by the liver. Little is known about the storage of protein, especially of protein stores in a form that is ready to be used. It is known that amino acids are needed for the processes of peptide synthesis and for protein repair within the body. Examples of protein synthesis in prior art assume the correct amino acids will be there, and they will be there just as they are needed.

Protein is found in collagen and muscle tissue but little further on the topic of short-term protein storage in the body can be discovered in the literature. In this disclosure, the lymph system will serve as the marshalling yard for amino acids utilized in peptide production within the body. The Lymph is directly connected to the brain via the spinal column. It therefore, is assumed to be directly under central nervous system control.

Free amino acids are accumulated in the lymph system, primarily, to serve bodily function in the synthesis of endocrine and neural hormones. Initial intake of amino acids into the lymphatic system is primarily accomplished through the intestines. If this, the secondary, or other sources of free amino acids are not available; the process of “robbing Peter to pay Paul” is used. Scavenger molecules are dispatched from lymph cells to collect amino acids from the break up of underutilized protein around the body. These individual carriers are specific for each needed amino acid. When that amino acid is located, it is transported back into the lymph system for a new, more pressing use.

Collagen is a common and frequently damaged protein in the human body and is among the first of the body's components to visually show damage. This damage to collagen is elsewhere said to herald the aging process. The skin of an aged face and body reflects damage by sun and other factors to the collagen supporting structure. Joints are held together by collagen and collagen plays an important role in tendons where loss of flexibility and range of motion first becomes apparent. Over time, collagen structures seem to decline and do not seem to obtain needed repair. As they become older, many individuals become noticeably shorter in stature. The disks separating the bones in the spine collapse as they, too, begin to lose collagen. The components of collagen are being taken away by the body to be utilized in other areas, such as in the production of peptides. The decline of collagen in the body is essentially part of the process of “robbing Peter to pay Paul.”

Striae or “stretch marks” are caused by defects in the collagen layer beneath the affected skin. Stretch marks usually occur during times of rapid growth or of rapid weight loss. Striae are caused by a sub-optimal amino acid uptake triggering the local unavailability, for repair purpose, of critical amino acids during a specific period of growth. Cellulite is another collagen defect due primarily to the local unavailability of a slightly different proportion of almost the same group of amino acids involving striae. Repair and improvement in these skin conditions can be affected with simple laser treatment. Successful and long-term therapeutic outcome must also involves the provision of the critical and missing amino acids in a form that can be taken up by the body.

The amino acid, proline is a major component of collagen. Depending on type, collagen is initially composed of from twenty to eighty percent of the amino acid proline. Proline, chemically, is an imino acid and the only imino acid used by the body to build peptides and structural protein. This physical and chemical difference is well documented within the literature. Proline is not considered to be an essential amino acid because it can be manufactured within the human body from lysine.

As one of twenty amino acids, proline is composing roughly five percent of amino acids in the average diet. Proline is necessary in very much greater percentages to build collagen. As a layer of collagen is visible beneath the skin, a shortage of proline may be among the first amino acid shortages to be visible. The addition of small amounts of proline to the diet of a young person may have good effect. The addition of small amounts of proline to the diet of an aged individual does not seem to show this same effect.

Proline has a major role in reproduction. Proline plays an important part in the maintenance of the lining of uterus, which is critical as the area of uterine contact with the placenta. High levels of proline allow the developing placental blood vessels to assume a “spring-like” structure. The incorporation of proline provides optimal blood flow by preventing or reducing the flattening of the blood vessel. The helical structure formed by proline rich vessels further cushions the developing fetus from physical shock that could tear away the developing connection. Older females are not easily able to provide the proline raw material to form those structures. They are not as physically able to carry a child, as they would have been if they were younger. This is why miscarriage is so common in older females.

Proline is an important part of reproduction for males, as well. Proline is a large and important component of semen. In fact, the standard tests for semen are actually simple chemical tests for the amino acid, proline. It has been shown that females can absorb the residual components of semen, including proline, through the vaginal membranes. This method of nutrient uptake may play a significant role in maintaining the, usually better, physical health of the female.

According to the present study, a 34 yo. female who had always reported painful periods now reports almost no pain, given proline in a form that can be absorbed. Having the right materials to correctly rebuild uterine walls appears to stop the formation of painful fibroids. The symptoms of painful fibroids have also been much reduced in a postmenopausal female by the addition of the missing nutrition in a form that may be absorbed.

This disclosure is both a method of maintenance of sufficient bodily reserves to be easily able to survive short-term disturbances and the interruption of food intake, as well as, the maximization of the health and wellness of an individual through the provision of good nutrition. Disclosed is the avoidance of health problems through improved nutrition, rather than methods to prevent genetically based health problems. Any genetic changes that may occur, or may not occur, and any effect such change may have, or may not have; on an individual's lifespan is expressly not a part of this disclosure.

This is not to say genetic mutations do not occur. True mutation occurs in the womb during the initial genetic sequencing of the individual. Mutations are not often observed because significant mutations are often incompatible with life. Not always readily observable, many smaller but important mutative changes can and will occur. This type of mutation becomes locked into the genetic code of the individual. Because it is compatible with survival, these mutations may then be passed along to that individual's future offspring. Certain and specific of these smaller genetic mutations are common and occur within all species. Most mutations are not successful and are lost to the species. However, any change in genetics that provides a significant edge to the mutant individual will soon dominate. Genetic changes that can provide robustness during the reproductive years are among the most genetically likely of changes to survive. Traits that are beneficial to survival during the reproductive years are therefore the most likely to be passed along. This type of mutation will readily be spread and come to dominate that individual species. It is in this manner, that a common and universally beneficial genetic change can become common to all species.

Genetic change that is “Front Loaded” to favor reproduction, even at the expense of longevity, will dominate. In 1970, it was described that the sexually active male Flatworm had a twenty-five percent lower longevity than non-sexually active males. Examples exist in nature, such as the male praying mantis, of “zero longevity” in preference of successful mating.

Successful strategies are genetically successful because the genes they represent are passed on to the next generation. This is true of the genetic mutation that is associated with the progression of the decline in nutritional uptake described in this disclosure. The bulk of the benefit of the one small change to a single gene in the digestive system goes to persons of reproductive age. In humans, the male and female both benefit, with females exceeding in benefit.

The number of species who share in a similar genetic change illustrates that these types of mutation are common. They are common because of the benefit to reproduction. This type of mutation has come to dominate the vast majority of the newer species. Only a few older species, such as the shark, do not share in this “short term” reproductive enhancement. The shark species has maintained its previous longevity by continuing to absorb nutrients in a steady and continuous fashion. Today, Great White sharks that are many hundreds of years old are relatively common in most of the world's oceans.

In humans, the trade-off for enhanced reproductive success is foreshortened lifespan. Advances in modern medicine have combined to make the opposite appear to be true. Yet, as we get older, the body becomes less efficient in the absorption of specific amino acids. The genetic exchange involved in an increased chance for species renewal, ultimately leads to decreased longevity of the individual.

Genetically, long-term maintenance of cells specific to the uptake of specific nutrients is sacrificed as to provide an earlier boost in the uptake of these nutrients. The uptake of a specific amino acid crucial to reproduction is maximized during that important period. A window of enhanced reproductive success is provided which will soon afterwards close. This short-term benefit was gained at the expense of long-term, continuous nutrient uptake.

The unique teaching of this disclosure is of differing pathways for the absorption of amino acids into the body and that the efficacy of these pathways may decline over time. The decline in the uptake of nutrients will result in specific and predictable events. Chief among these is a decline in longevity. There is a natural, species-specific nutrient uptake limit. However, there is also genetic variability among individuals in the absolute uptake of nutrient. As illuminated in the Claims section of this document there are various methods useful in dealing with a disparity of uptake.

The example of this disclosure is the amino acid, proline. Proline will not always absorb well through the gut. Until now, science seems to have assumed that proline is absorbed into the body in the same way as are the rest of the amino acids. Certainly, no one has before suggested there may a biological limit on the uptake of nutrients. Even researchers whose data indicates there may be a problem with proline uptake continue to follow the orthodoxy and ascribe a genetic cause. Fortunately, the case for a lack of nutrient uptake can be made, and documented.

Proline is an amino acid soluble in alcohol. This explains longevity found in cultures where wine is always served with a meal. Alcohol is absorbed through the stomach, not the intestine. Proline, from partially digested protein, can enter the body in small amounts through the stomach, dissolved as it were, in ingested alcohol. In this way, proline is still able to be absorbed even when it no longer can be absorbed normally in the intestines.

In the present study, active exercise buffs and body builders report success when provided with proline solubilized in alcohol at a twenty-five gram daily dose. One report is quoted; “I have found over the past 8 or 9 months of consistent use, that my energy, strength, and short term endurance have dramatically increased. I've also found that I have significantly faster recovery between sets during a weight lifting workout, as well as complete recovery between day-to-day workouts. I have also noticed an increase in lean muscle mass over this period of time.” Strength building exercises involve breaking muscle cells as to have muscles rebuild in a stronger fashion. The object is to increase the bulk of the muscles by eukaryotically increasing the number of cells.

Adding additional quantities of the scarce component to the diet keeps the body from competing for it with the now broken down cells. Further, not having to stop the rebuilding process to rob the scarce component from other cells increases overall resilience. Having a ready access to the missing amino acids rather than having to hunt for them allows these cells, and the body, to recover faster from exercise.

The human body is a masterpiece of repair and renewal. It is well known and elsewhere discussed that dead cells show evidence of oxidative damage. This led to the general conclusion that anti-oxidants may be helpful in preventing cell aging. Oxidation may not be the cause of cell death. Instead, oxidation may simply be the result of the cell's decline. The proximal cause of most bodily cell death is the individual cells do not get the signal for cellular repair. The individual cell was likely targeted for cellular death to free its scarce amino acids for other use.

It takes sixty days for a human body to starve to death, yet only a week for it to die from the lack of water. This physical and nutritional resiliency is due to the adaptive reuse of available bodily resources. Much of the body's power to reconfigure itself is due to the intricacies of the lymphatic system. Among other uses, the lymph system serves as the marshalling yard for amino acids. Specialized carriers formed in the lymph nodes are transporters of specific amino acids. It is their role to insure that specific amino acids are available exactly when needed.

It was necessary to puzzle-out the lymph system's role in survival during shortage to identify the processes used to keep older persons alive if they are not absorbing the nutrition needed for normal function. As we get older, the process of maintaining longevity is basically the same as the prolongation of life occurring during starvation. This can be stated another way. A lack of nutrition due to a long-term decrease in the uptake of a few amino acids is simply a slowed progression of the same natural course of events found in outright starvation. What is broadly called “aging” is a collection of symptoms. Included in these symptoms is the lack of longevity. Lack of longevity is an elongated process, a modified form of continuing starvation. Lack of longevity becomes failure to thrive.

The lymph system plays an important role in survival during times of famine. The lymphatic system collects, transports, holds, and recycles critical amino acids. This recycling function, especially for the amino acids that are in short supply, keeps the bodies signaling, immune and specific repair systems functioning. The object of cellular recycling is to keep the body alive during times of shortage. This function is under the direct control of the central nervous system. Therefore, the attitude, emotions, and neural hormone levels of the individual play an important role in the evolving outcome. If you think things will get better, you will (more than likely) make it until they do.

This detailed description of the invention is not primarily concerned with how the body uses peptides, instead to emphasize that peptides will almost always be in production. Peptides, depending on size, will contain all of the twenty letters of the amino acid alphabet. Essentially, all twenty amino acids are in constant need in order to maintain peptide production. These amino acids are further required in an exact order, quantity and timing to form signaling peptides. This is an important point. Lack of a single amino acid, at the critical moment, is enough to shut down the neural peptide production system, as well as, those bodily feedback loops associated with the peptide. This requirement for a continuous supply of amino acids holds true for all orders of cellular organisms, including those bacteria living in the gut of a higher organism.

Until the work of Bonnie Brassier, science assumed that bacteria living in our gut did little more than to help us to digest our food. Brassier showed that bacteria produce quorum sensing peptides for both species-specific and intra species communication. Production of these peptides boosts bacterial amino acid requirements above the minimum requirement earlier researchers anticipated for cell maintenance and growth. Neural hormone peptide synthesis further boosts the need for specific amino acids to be available in a more timely fashion as compared to the need for simple availability as in the production of structural protein.

Gut bacteria and the individual cells of the human host are intertwined in the story of the survival of that individual. There are similarities and differences. Bacteria are single cell organisms that grow until they become large enough to split into two cells; and the cycle continues. The human body constantly has cell that are growing and some cells to die. In the human body, nothing (good) happens unless something tells it to happen. Communication between cells and systems of the human body is a continuous feedback loop driven by peptide production. Essentially, it is simply a more complex version of bacterial quorum sensing.

Gut bacteria are good indicators of the condition of the body as a whole. It is generally expected that an individual's stools tend to get weaker as their body ages. This phenomenon occurs in spite of the fact that the bacteria in the gut are receiving more of some nutrients. The species-specific uptake limit for protein is, somehow, negatively affecting gut bacteria. Younger persons have large quantities of bacteria in their stools. Older persons, over time, usually have less and less bacteria in their stools. The decline in bacterial content from the stools of younger persons to the stools of older persons is quite significant. Again, the process is viewed as “normal aging.” Proving the existence of a species-specific uptake limit involves illustrating how this process can be harmful to the bacteria in the gut.

The species-specific uptake limit on higher animals makes their gut bacteria the beneficiaries of the five amino acids that become increasingly harder for the aging body to uptake. These extra amino acids will simply remain in the gut of the higher animal, as the intestines do not well absorb them. It is not hard to picture an increase in relative concentration of these amino acids in the total mix. Over time, as the uptake of the other amino acids is accomplished, the concentration of these amino acids continues to increase. Finally, it is not hard to imagine that eventually these will be the only free amino acids available in the gut.

Bacteria are not selective eaters. They absorb and utilize nutrients in the immediate vicinity. The nutrients available to them in the human gut are normally the exact combination and concentration necessary for them to flourish. This changes in the aging body. Eventually, the unabsorbed amino acids will become the only amino acids available in the gut of the aged human. Dutifully, the gut bacteria will absorb and begin incorporating these amino acids, if they can.

To the bacteria living in the gut of the aged human, the extra five amino acids soon becomes a minus fifteen of the unsupplied amino acids. The additional quantity of those supplied amino acids creates a shortage of the ones not supplied. Because all twenty amino acids are essential for cellular processes in roughly the same amount, adding extra of any five creates a de facto shortage of the other fifteen. What appears to be extra, on one hand, becomes a net shortage in the other.

Bacterial peptides and processes that were begun using those additionally supplied amino acids cannot, now, be easily and timely completed. The system slows to a standstill due to what has become a relative shortage of the balance of the necessary amino acids. The remaining amino acids essential for the completion of the peptide are now in short supply. Other bacteria are competing for these scarce resources, as well. Bacterial peptide production and processes are constantly being halted in the early stages of production. Production of bacterial protein can go no further until a specific amino acid arrives. The bacteria are starved until additional, specific amino acids are made available.

Adding additional amino acids of the type well absorbed in the aged human body largely reverses this phenomenon. Providing extra quantities of these missing fifteen amino acids reverses the decline in the number of gut bacteria. As little as a gram of each of the fifteen amino acids taken daily can make a huge difference in the bacterial population in the gut. In multiple individuals, adding thirty grams of the combined mixture to the diet made a huge difference. This change in the nutritional uptake of an older person restored their stools to approximate the bacterial content of a much younger person. That this work-around is successful adds further evidence of the validity of the species-specific uptake limit.

The gut bacteria, E. coli is responsible for ninety-five percent of all urinary tract infections. Most of these infections are lower urinary tract or bladder infections. Kidney or upper urinary infections occur less frequently but are much more severe. The majority of each type infection occurs in females. Many older females are plagued with chronic urinary tract infections. The same bacteria cause both lower and upper urinary tract, or cause both bladder and kidney infections. Why is upper urinary tract infection so much worse?

Urine contains amino acids. However, urine is known to not contain the pure amino acid proline. Some salts are excreted but not the amino acid. Bacteria, especially an E. coli sample from an individual in their late twenty's or older, incorporate extra amounts of proline. A shortage of proline would not affect these bacteria as quickly as it would affect other bacteria. E. coli can live in the human bladder, if not thrive. If E. coli does make it to the kidneys, all amino acids are suddenly available to them and their population explodes. Clearing infection from a normally hosted bacterium is an almost impossible job for the immune system. Due to incorporation of proline in adult grown bacteria, E. coli infections often become a chronic problem. The problem becomes common because of the effect on them of the species-specific nutrient uptake limit of their host.

Wasting diseases such as cancer are characterized by apoptotic-like cell death. This is actually the rapid break down of immediately unneeded cells to provide nutrients scarce to bodily and immune response processes and to fuel the cell growth triggered by the disease. The more common amino acids, excess to this need, are simply discarded. They are found to clog the kidneys and become the cause of kidney failure. The administration of the now “formerly scarce” amino acids reverses this phenomenon. An adult Stage IV esophageal cancer patient given 30 grams of this supplement daily rapidly stabilized in weight and did not develop kidney failure. Provision of needed nutrition, in a form that may be absorbed, stops programmed cell death. If cellular death is reduced, the kidneys do not become clogged and damaged with the remainder amino acids.

This is a reason childhood cancer patients can have a better prognosis than adult cancer patients. Children still absorb all of the amino acids. They do not have need for cells to rob other cells to obtain that which their body needs to survive. Children can obtain complete nutrition from their diet. Verses adults, especially older adults, they “save a step” in the production of immune cells.

Pulmonary hypertension is a disease characterized by the loss of collagen from the walls of very small arterioles in the lung. Because of their small size, the arterioles are especially vulnerable to scavengers seeking to rob them of critical nutrients. Without firm walls lining the vessel they balloon outward causing the body to increase arteriole pressure to effect blood flow. Over time, the condition is made worse because vessel wall amino acids are now further exposed to “amino-acid robbery.” Addition of the missing amino acid to the diet readily improves the condition. In fact, the increased arteriole exposure seems to have made critical amino acid re-addition more easily possible. In the one trial, a dose of thirty grams of alcohol solubilized proline, daily, quickly caused substantial clinical improvement in the condition.

Changes made to the body through changes in nutrition can affect the course of many disease processes. Infectious agents, such as viruses and the course of diseases they cause may also be affected. The herpes virus becomes dormant by attaching its cell coat to the dorsal-root ganglia rather than becoming fully integrated into host cells, as do most viruses. This is beyond the blood-brain barrier and normally out of the reach of the immune system. This ability to hide is what keeps the viruses from being eliminated from the body. While dormant, herpes expresses few genes but are known to detect relative circulating levels of lysine, arginine, and ornithine High levels of lysine, relative to other amino acids has been shown to shorten herpes infection. This has also been shown to keep herpes viruses dormant. This is why lysine is commonly sold as an aid to prevent fever blisters.

Low relative levels of lysine has for thousands of years been a reliable signal to the virus that the host immune system is busy elsewhere. Because the body only converts lysine to proline in extreme circumstance, high circulating levels of proline becomes a signal for the virus to detach from the ganglia. Perhaps because they attach to high proline areas of the ganglion, Herpes will then almost automatically detach, become active and travel to areas where they more easily can be transmitted again.

Herpes has the unique ability to move much more quickly than other viruses because they do not fully enter a host cell to hide. The process of Co-evolution has refined their ability to come and to go quickly and reliably. Detection of increased circulating lysine tells them when they need to hide. This system has worked well for them as long as humans have come together to live in groups.

Now, we may easily alter blood levels of proline by sharply increasing its intake. We can provoke the Herpes virus to come out of hiding. Only this time, the virus will be forced to go against an alert and much stronger immune system. High proline levels used to trigger a herpes attack must remain high to successfully prevent the virus from retreating. One trial was done at ten times the normal daily dose of proline solubilized in alcohol given for a period of seven days. With the addition of acyclovir, the virus appears to have been successfully cleared from the body.

Related to this are the increasingly common occurrences of the diseases HIV/AIDS and Multiple Sclerosis. Herpes infection may be behind the disease process of both. Human Immunodeficiency Virus does not trigger the immune system, but must have an on-going active immune process to be able survive and to transmit itself. Herpes may just be the active co-partner that HIV has evolved into needing. When “dormant,” herpes does not go dormant like other viruses. Herpes differs by not becoming incorporated into host cells. Herpes is perhaps only semi-dormant in hiding to be prepared to strike quickly if they are again triggered. During the time immediately after an active herpes infection, the immune system is fully aware of the presence of the virus. It simply is not able to get to the virus because of the blood brain barrier. This continuing awareness of the virus keeps the immune system active and replicating long after the initial response. This extended and fully engaged immune response gives additional opportunity, and certainly opportunity in the correct location, size, and scope for HIV to take advantage.

Multiple Sclerosis may be another process to be “the beneficiary” of herpes. Multiple Sclerosis is an autoimmune process, whereby the immune system mistakenly targets components of the central nervous system. Likely originating by immune cells passing through gaps in the blood brain barrier, the attacks originally seem to involve areas where herpes has been physically connected to the ganglia. Binding to nerve cells somehow can leave enough viral coat to trigger an immune response against these cells. Whatever the exact trigger, it causes great harm to the individual. The episodic nature of the disease with acute, remission and relapsing remission phases sometimes lasting for a year evokes comparison with the extended immune system response to herpes. The immune system seems to respond far beyond what would be a normal response to the active part of the herpes infection. In any case, active remission could be accomplished by ridding the body of the herpes virus by the use of the above process.

Pure powder (free) amino acids are used in all preparations of L-proline in this disclosure. Proline is readily soluble in alcohol. Proline in alcohol freely enters the blood stream by absorbing through walls of the stomach. This method, taken nightly, insures the absorption of an amino acid that is not always absorbed well through the intestines.

It has been found that one hundred proof vodka is the most convenient and readily available vehicle for this use. Tax paid distilled vodka gives great additional flexibility in the use of the finished product. L-proline is used because of its biological activity. L-proline is slightly sweet and adds only the slightest pleasant taste to the vodka. Other than the alcohol being diluted, by volume, it may be treated as one would treat regular vodka. The product may further be diluted with juice or soft drink for final consumption. Alcoholic or hydro alcoholic solutions are preferred in this use.

The current standard of preparation is one-half cup of pure L-proline powder added in two portions to one cup of vodka, with aggressive agitation. This mixture is highly stable and can tolerate excursions in excess to the extremes found in year-round storage in the average warehouse. It has been suggested that pure L-proline powder become a regulated substance due to the potential of abuse with self-medication.

In operation, the dietary supplements disclosed may further include a food additive. The term food additive as used herein may refer to a flavoring, a colorant, a stabilizer, or a texture additive. Flavorings that may be added to the dietary supplement may include sweeteners, which may be artificial. Other natural flavorings, such as mint or fruit flavors, may be added to the dietary supplement. Any known colorant may be used to color the dietary supplement in order to increase visual appeal. Many colorants used in food and beverage products are well known in the art, and include colorants such as Allura Red AC.

The food additive may also be a texture additive. The texture additive may be a solid, such as another food. The dietary supplement may be used in combination with any other type of food, such as brownies or granola bars. Alternatively, the dietary supplement may be served as a beverage, and the texture additive may include additives such as pulp or carbonated water.

The food additive may include stabilizers. Stabilizers may be used to adjust factors such as the acidity and shelf life of the dietary supplement. For instance, stabilizers may include sorbic acid, ascorbic acid, sulfur dioxide, sodium benzoate and mixtures thereof.

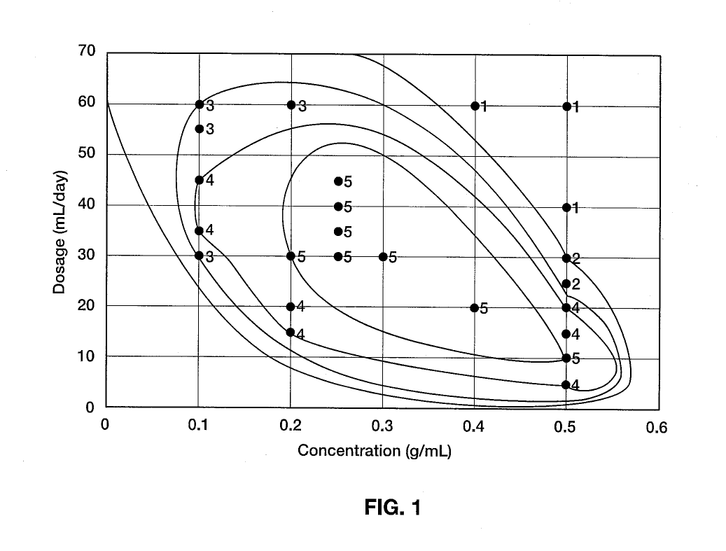

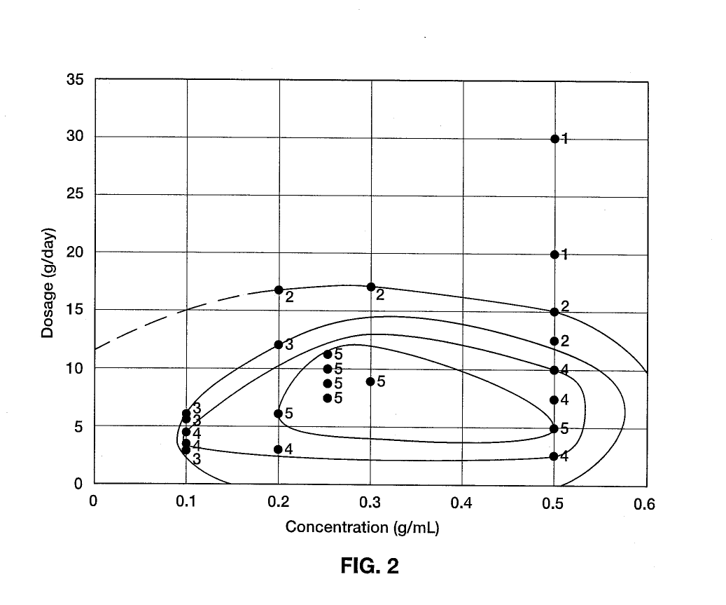

Referring now to the drawings in general, it will be understood that the illustrations are for the purpose of describing a preferred embodiment of the inventions and are not intended to limit the inventions thereto. FIG. 1 illustrates the desired relationship between the dosage administered (in milliliters per day) and concentration (in grams of amino acid per milliliter of solvent) via a two-dimensional surface plot. FIG. 2 illustrates the desired relationship between normalized dosage (in grams per day) and concentration (in grams of amino acid per milliliter of solvent) via a two-dimensional surface plot. Various concentrations at various dosages are plotted and ranked from 1 to 5 indicating least to most desirable in terms of positive and negative effects. The response surface shown by each figure provides a visual illustration of the effects of both dosage and concentration via a two-dimensional surface plot of a three-dimensional surface. The boundary conditions in FIGS. 1 and 2 are denoted with lines, and indicate the dosages and concentrations ideal for use in the present inventions.

Accordingly, data was plotted based on observed positive and negative effects from patients taking a dietary supplement of proline dissolved in ethanol at a particular concentration and dosage. The observations reported from these studies are discussed below. FIGS. 1 and 2 indicate that the most effective concentrations of the dietary supplement has a concentration of about 0.2 and about 0.4 grams of proline per milliliter of ethanol, with about 0.3 g/mL being the most effective. However, various other concentrations between about 0.1 g/mL and 0.5 g/mL were found to have some efficacy depending upon dosage. Both graphs indicate that excess dosages of the dietary composition do not lead to greater efficacy, but instead introduce negative side effects that pose a limit to the effective amount.

Turning to FIG. 1, the response surface indicates that the effective volume dosage (e.g., milliliters per day) is highly concentration dependent. At lower concentrations of the dietary supplement, the most effective volume dosages lies between about 30 milliliters to about 50 milliliters per day. At higher concentrations, the most effective volume dosages lies between about 10 milliliters to about 20 milliliters per day.

The effective volume dosages at each concentration do not appear to share the same gram amounts of proline consumed per concentration. FIG. 2 is a response surface that highlights this behavior of the dietary supplement. The data indicates that the effective gram amount of proline varies according to the concentration of the dietary supplement. At a concentration of about 0.25 g/mL, the effective gram amount of proline is between about 5 grams to about 12 grams per day. At other concentrations of the supplement, consuming the same gram amount of proline does not necessarily lead to the same level of efficacy. At about 0.1 g/mL, none of the dosages display the same efficacy as shown with the concentrations between about 0.2 g/mL and about 0.3 g/mL. Similarly, at about 0.5 g/mL, lower amounts of proline are more effective than those observed at lower concentrations of the supplement. At all concentrations, exceeding 15 grams per day leads to negative side effects that outweigh the positive benefits of consuming the dietary supplement.

These graphs show that efficacy is not solely dependent on the grams of proline consumed, but rather, that efficacy is also dependent with respect to the concentration of proline dissolved in a particular biocompatible solvent. The results indicate that it is the combination of the solvent and proline responsible for the positive effects reported herein, and that there are ideal ranges of concentration and dosages for the combination. The positive effects observed is not based on each component alone, as seen with the non-linear dependency of effective proline amount with respect to concentration.

Various positive effects were seen to be attributable to consumption of the dietary supplements disclosed above. Quality of life assessments were given to individuals in several clinical studies. Quality of life was rated as 100% improved in 100% of the subjects by the end of the two-weeks in one clinical study. Individuals displayed more confidence and reported feeling and functioning better than prior to consumption of the dietary supplement. Some of the health benefits and improvement in health shown in these studies include: increased energy and stamina, improved appearance of the skin, increased muscle mass, increased immune function and the reversal of autoimmune disorders, as well as, the resolution or abatement of many long-term illnesses.

Various negative effects were also seen in some patients, particularly at higher dosages. Some of the negative effects reported include:
High levels of high concentration solution stimulated Human Papilloma Virus (flat wart) growth on the face and neck.
High concentrations of alcohol (irrespective of the concentration of active ingredient) caused problems with medication, drug interactions and inebriation.
Dry itchy skin, burning, itching, pain, and sinus pressure at higher doses of lower concentration.
Non-productive coughing associated at. higher doses of the weaker concentration product.
Difficulty breathing and respiratory symptoms such as orthopnea were reported in ingesting lower concentration but not in higher concentrations. This unexpected effect may be dehydration related.
Extremely high dosage levels of the highest concentration product have caused herpes outbreaks.

The triggering of latent viral infections was the most significant of the unexpected properties of proline that was discovered during our trials. That this occurred at higher doses uncovered the potential trigger mechanism of viral attack. The reported observed effects seen at extremely high doses where herpes outbreaks were triggered may lead to future studies aimed at preventing autoimmune and other diseases related to herpes infections.

In at least one study, proline is shown to be well absorbed in healthy volunteers. Other sources state proline is not well absorbed or simply assume that oral absorption is unimportant. In trials with older subjects (over the age of 40) and the current proline product, positive effects were achieved via supplementation in all cases. Proline is one of twenty amino acids used in the body to form protein. Per the vast majority of the prior art: proline is considered as non-essential. The current trial proves proline to be very much essential in older individuals, especially. Additional sources state proline may be “conditionally essential—in certain disease states.” As seen by the positive effects reported herein, simply because the body can synthesize an amino acid does not exclude the need for its supplementation.

Certain modifications and improvements will occur to those skilled in the art upon a reading of the foregoing description. By way of example, the dietary supplement may be consumed as a beverage or food item. In certain aspects, the dissolved mixture may be mixed with any other type of consumable liquid such as carbonated water. It should be understood that all such modifications and improvements have been deleted herein for the sake of conciseness and readability, but are properly within the scope of the following claims.

What is claimed:

1. A composition for a dietary supplement comprising an effective amount of amino acid and a biocompatible solvent.

2. The dietary supplement of claim 1 further including a food additive.

3. The dietary supplement of claim 2, wherein the food additive is a flavoring.

4. The dietary supplement of claim 3, wherein the flavoring is a sweetener.

5. The dietary supplement of claim 4, wherein the sweetener is an artificial sweetener.

6. The dietary supplement of claim 2, wherein the food additive is a colorant.

7. The dietary supplement of claim 6, wherein the colorant is Allura Red AC.

8. The dietary supplement of claim 2, wherein the food additive is a texture additive.

9. The dietary supplement of claim 8, wherein the texture additive is a solid.

10. The dietary supplement of claim 9, wherein the solid is another food.

11. The dietary supplement of claim 10, wherein another food is pulp.

12. The dietary supplement of claim 8, wherein the texture additive is a liquid.

13. The dietary supplement of claim 12, wherein the liquid is carbonated water.

14. The dietary supplement of claim 2 further including stabilizers.

15. The dietary supplement of claim 14, wherein the stabilizers are selected from the group consisting of sorbic acid, ascorbic acid, sulfur dioxide, sodium benzoate and mixtures thereof.

16. A composition for a dietary supplement comprising an effective amount of amino acid and a biocompatible solvent, wherein the effective amounts of amino acid is between about 1 gram per day and about 20 grams per day.

17. The dietary supplement of claim 16, wherein the effective amount of amino acid is between about 2 grams and about 15 grams per day.

18. The dietary supplement of claim 17, wherein the effective amount of amino acid is about 10 grams per day.

19. The dietary supplement of claim 16, wherein the amino acid is L-proline.

20. The dietary supplement of claim 16, wherein the biocompatible solvent is a polar organic solvent.

21. The dietary supplement of claim 20, wherein the polar organic solvent is an alcohol.

22. The dietary supplement of claim 21, wherein the alcohol is ethyl alcohol.

23. The dietary supplement of claim 16, wherein the concentration of amino acid, in a biocompatible solvent, is between about 0.1 and about 0.5 grams per milliliter.

24. The dietary supplement of claim 23, wherein the effective concentration of amino acid in a biocompatible solvent is between about 0.2 and about 0.4 grams per milliliter.

25. The dietary supplement of claim 24, wherein the effective concentration of amino acid in a biocompatible solvent is about 0.3 grams per milliliter.

26. The dietary supplement of claim 16 further including the balance water.

27. The dietary supplement of claim 26, wherein the balance water is more than about 50 vol. %.

28. The dietary supplement of claim 27, wherein the concentration of water is about 60 vol. %.

29. A composition for a dietary supplement comprising:
(a) an effective amount of amino acid and a biocompatible solvent, wherein the effective amounts of amino acid is between about 1 gram per day and about 20 grams per day; and
(b) a food additive.

30. The dietary supplement of claim 29, wherein the food additive is a flavoring.

31. The dietary supplement of claim 30, wherein the flavoring is a sweetener.

32. The dietary supplement of claim 31, wherein the sweetener is an artificial sweetener.

33. The dietary supplement of claim 29, wherein the food additive is a colorant.

34. The dietary supplement of claim 33, wherein the colorant is Allura Red AC.

35. The dietary supplement of claim 29, wherein the food additive is a texture additive.

36. The dietary supplement of claim 35, wherein the texture additive is a solid.

37. The dietary supplement of claim 36, wherein the solid is another food.

38. The dietary supplement of claim 37, wherein another food is pulp.

39. The dietary supplement of claim 35, wherein the texture additive is a liquid.

40. The dietary supplement of claim 39, wherein the liquid is carbonated water.

41. The dietary supplement of claim 29 further including stabilizers.

42. The dietary supplement of claim 41, wherein the stabilizers are selected from the group consisting of sorbic acid, ascorbic acid, sulfur dioxide, sodium benzoate and mixtures thereof.

43. The dietary supplement of claim 29, wherein the effective amount of amino acid is between about 2 grams and about 15 grams per day.

44. The dietary supplement of claim 43, wherein the effective amount of amino acid is about 10 grams per day.

45. The dietary supplement of claim 29, wherein the amino acid is L-proline.

46. The dietary supplement of claim 29, wherein the biocompatible solvent is a polar organic solvent.

47. The dietary supplement of claim 46, wherein the polar organic solvent is an alcohol.

48. The dietary supplement of claim 47, wherein the alcohol is ethyl alcohol.

49. The dietary supplement of claim 29, wherein the concentration of amino acid, in a biocompatible solvent, is between about 0.1 and about 0.5 grams per milliliter.

50. The dietary supplement of claim 49, wherein the effective concentration of amino acid in a biocompatible solvent is between about 0.2 and about 0.4 grams per milliliter.

51. The dietary supplement of claim 50, wherein the effective concentration of amino acid in a biocompatible solvent is about 0.3 grams per milliliter.

52. The dietary supplement of claim 29 further including the balance water.

53. The dietary supplement of claim 52, wherein the balance water is more than about 50 vol. %.

54. The dietary supplement of claim 53, wherein the concentration of water is about 60 vol. %.
